(19) United States
(12) Patent Application Publication
(10) Pub. No.: US 2014/0088703 A1
(43) Pub. Date: Mar. 27, 2014

(54) SOFT PROSTHETIC IMPLANT MANUFACTURING PROCESS

(71) Applicant: Allergan, Inc., Irvine, CA (US)

(72) Inventor: David Schuessler, Ventura, CA (US)

(73) Assignee: Allergan, Inc., Irvine, CA (US)

(21) Appl. No.: 14/095,892

(22) Filed: Dec. 3, 2013

Related U.S. Application Data
(63) Continuation of application No. 13/128,497, filed on Jun. 22, 2011.
(60) Provisional application No. 61/113,504, filed on Nov. 11, 2008.

(51) Int. Cl. A61F 2/12

(52) U.S. Cl. CPC A61F 2/12
USPC 623/8; 264/255

(57) ABSTRACT

A soft prosthetic implant, for example, a breast implant, having a shell filled with a fluid. One or more components of the soft prosthetic implant receive a unique identifier for traceability during the manufacturing process. The unique identifier is placed on one component, such as a patch molded-in-place with a shell to form a soft prosthetic implant. Information about the respective components, as well as process parameters, can be stored on the unique identifier for later reference. The unique identifier may be a two-dimensional printed label or laser-etched characters, or may be a three-dimensional embossed or indented region. The unique identifier may be a separate label attached to the respective component, or may be formed in the component, such as with embossing.

### CROSS REFERENCE

This application is a continuation application of U.S. patent application Ser. No. 13/128,497, filed on May 10, 2011, which is a national stage application under 35 U.S.C. §371 of PCT Patent Application No. PCT/US2009/063606, filed on Nov. 6, 2009, which claims the benefit of U.S. Provisional Patent Application No. 61/113,504, filed on Nov. 11, 2008, the entire disclosure of each of these applications being incorporated herein by this specific reference.

### FIELD OF THE INVENTION

The present invention relates to manufacturing processes for prosthetic implants and, more particularly, to a method of tracking an individual soft prosthetic implant component during its manufacturing history.

### BACKGROUND OF THE INVENTION

Soft implantable prostheses are commonly used to replace or augment body tissue. In the case of breast cancer, it is sometimes necessary to remove some or all of the mammary gland and surrounding tissue. This creates a void that can be filled with an implantable prosthesis. The soft implant serves to support surrounding tissue and to maintain the appearance of the body. The restoration of the normal appearance of the body has an extremely beneficial psychological effect on post-operative patients, eliminating much of the shock and depression that often follows extensive surgical procedures. Implantable prostheses are also used more generally for restoring the normal appearance of soft tissue in various other areas of the body.

Soft implantable prostheses typically include a relatively thin and flexible envelope or shell molded from silicone elastomer which is then vulcanized (cured). The shell is filled either with a fluid such as a silicone gel or a physiologic saline solution. Filling of the shell takes place before or after the shell is inserted through an incision. The present invention pertains to any type of fluid-filled prosthesis, but is especially beneficial for use with gel-filled shells.

A conventional dip-molding process for forming flexible implant shells for prostheses involves dipping a suitably shaped mandrel into a silicone elastomer dispersion. The shell is peeled from the mandrel and a shell hole resulting from the molding process is patched. The hollow interior of the shell is then filled with an appropriate filling material, for example a silicone gel, by means of an aperture in the patch. The aperture in the patch is then sealed with a silicone adhesive and the prosthesis is heat cured.

Another process for forming implant shells is rotational molding, such as the system and methods described in Schuessler, U.S. Pat. No. 6,602,452, the entire disclosure of which is incorporated herein by this reference.

Saline filled breast implants are typically constructed of an outer shell of several layers of silicone rubber and a valve. Saline implants are usually implanted in the breast cavity empty or only partially filled, and then inflated to their final size by one or more syringes or a disposable tube connected to a saline reservoir.

After manufacture, a batch of prostheses is labeled and packaged. Before packaging however, unfilled shells often remain unlabeled and in storage for a significant time before each shell is removed from its respective batch and further processed to create the final, assembled prosthesis. Although the assembled product itself may be labeled with identification information, each individual component of the implant is not easily traceable to its point of origin.

There is a need for processes that provide enhanced transparency about specific steps in the manufacture of individual prostheses, so that potential problems, for example, can be traced back to their origin and corrected.

### SUMMARY OF THE INVENTION

In accordance with the invention, a process for forming a prosthesis, for example, a soft implant, for example, a breast implant, (hereinafter sometimes simply “implant”) is provided, the process generally comprising providing a fluid dispersion and molding the dispersion to form a hollow envelope or shell having an internal cavity. The cavity can subsequently be filled with a suitable filler material, for example, saline or silicone gel. The shell, with or without filling material, makes up the implant.

In accordance with one aspect of the invention, the process further comprises the step of providing an identifier, for example, indicia, on one or more components of the implant during the manufacture of that component. For example, an identifier is provided on the shell during the manufacture thereof, for example, during the steps of molding the shell. In one embodiment, the identifier is unique to the specific breast implant relative to other implants, even those other implants that have been made at the same time and/or in the same batch. The identifier remains with the shell during each subsequent implant manufacturing step and can be used to facilitate tracing the manufacturing history of the shell back to its original formation.

In one embodiment, the identifier is provided on the implant by providing a secondary component which includes the identifier, and securing the secondary component to the shell. The secondary component is secured to the shell during the process of molding the shell. For example, in accordance with one embodiment, the step of providing an identifier comprises forming a label or patch having the identifier thereon, and molding the label or patch to the shell, for example, while the shell itself is being molded. In one embodiment, the label or patch bearing the identifier is molded into the shell, for example, embedded between material layers which make up the shell.

In another aspect of the invention, a process for forming a soft implant generally comprises providing a starter material and forming the material into a first component; providing a second component including an identifier, and assembling the first and second component to form the implant having the identifier. In some embodiments, a second identifier is also provided, for example, on the first component, the second identifier being different from the first identifier. In addition, a third identifier may be provided on the assembled implant, the third identifier being different from the first and the second identifiers.

The starter material may comprise an elastomeric material, for example, a silicone elastomer. The step of forming the elastomeric material into a first soft component comprises one of dip-molding or rotational molding the material, for example, a dispersion of the material, on a molding surface, for example, a mandrel, thereby forming a flexible, fillable elastomeric shell. In a specific embodiment, the step of providing an identifier includes forming the identifier on the shell by molding the identifier into the shell during the dip molding or rotational molding of the shell itself, for example, by providing a negative imprint of the identifier on a molding surface, for example, the mandrel, for the shell.

In another aspect of the invention, a process for facilitating tracking one or more components of an implant, for example, a breast implant, is provided. In a specific embodiment, the process comprises forming components of a breast implant, providing an identifier on one or more of the components during the process of forming the components, assembling the components with one another to form an implant. In a specific embodiment, the process comprises the steps of molding an elastomeric shell having an internal cavity, providing a patch having a unique identifier thereon, and covering a hole in the shell with the patch to form a fillable breast implant. In one embodiment, one or more parameters of the breast implant manufacturing process are recorded and linked with the identifier. The recorded parameters may include information relating to the chemical and physical makeup of the shell, the date of manufacture, the place of manufacture, as well as other useful information.

### BRIEF DESCRIPTION OF THE DRAWINGS

Features and advantages of the present invention will be appreciated as the same become better understood with reference to the specification, claims, and appended drawings wherein:

FIGS. 1A-1C show several steps in a process of dip-forming the shell of a breast implant prosthesis, wherein a dip mold features a unique identifier label for transfer to the formed shell;

FIG. 2 is a cross-section of an exemplary filled breast implant prosthesis having a unique identifier label thereon;

FIG. 3 is a cross-sectional view through an alternative filled breast implant shell showing an exterior shell identifier and a second identifier label provided on a sealing patch;

FIG. 4 is a cross-sectional view through one portion of a smooth-walled breast implant prosthesis shell having an identifier label embedded between layers of the shell;

FIG. 5 is a cross-sectional view through one portion of an alternative breast implant prosthesis shell having an identifier label embedded below surface texturing;

FIGS. 6 and 7 are top plan and vertical sectional views of an exemplary mold for a rotational molding system having a unique identifier within a mold cavity;

FIG. 8 is a cross-sectional view through an exemplary gel-filled breast implant prosthesis having a molded-in-place flush patch having a unique identifier in accordance with the present invention;

FIG. 9A is a detailed view of the interface between the flush patch with the unique identifier and the shell of the breast implant prosthesis of FIG. 8;

FIG. 9B is a detailed view of the interface between an alternative flush patch with the unique identifier and the shell of the breast implant prosthesis of FIG. 8;

FIG. 10A is a flow chart illustrating an exemplary process for applying a unique identifier label to a soft implant prosthesis during manufacture;

FIG. 10B is a flow chart illustrating one process for applying a unique identifier label to a soft fluid-filled implant prosthesis during manufacture; and

FIG. 10C is a flow chart illustrating another process for providing a unique identifier label on a soft fluid-filled implant prosthesis during manufacture.

### DETAILED DESCRIPTION

The present application relates to assembled medical prosthetic implants. In accordance with a broad aspect of the invention, manufacturing processes are provided whereby the history of an assembled implant can be tracked or traced, for example, uniquely traced, to early points of manufacture.

In accordance with one embodiment of the invention, manufacturing processes generally comprise forming a component of an implant assembly and labeling the component with an identifier, for example, a unique identifier, for example, unique indicia specific to that particular component, during an early stage in the formation of the component. More specifically, the process may comprise forming a shell of a breast implant and labeling the shell with a unique identifier during molding of the shell. The identifier may comprise, for example, a serial number, bar code, indicia, transponder, or other means for human or machine-readable output. Consequently, in some embodiments, the unique identifier becomes integrated into an assembled breast implant including the shell, thereby facilitating tracing of the specific shell.

This aspect of the present invention can be contrasted with conventional manufacturing processes which place identification information on preformed or pre-molded shells that are each part of a batch of such shells, each shell not being individually distinguished from each other. Such shells remain unlabeled and in storage for a significant time before each shell is removed from the batch and further processed to make an assembled implant.

Although the assembled implant product itself may be labeled with identification information, each individual component of the implant is not easily traceable to its point of origin.

A shell of an implant in accordance with the invention is a flexible, elastomeric component that is typically dip-molded or rotational molded by applying a starter material, for example, an elastomeric material in a dispersion form, to a mandrel having the shape of the implant. Due to the nature of forming such a shell, a mandrel hole or sprue hole remains in the shell after the shell is removed from the mandrel. Prior to filling the shell with a filler material, such as silicone gel or saline, the hole is sealed with a patch. In one embodiment of the invention, the unique identifier is provided on the patch and the patch is molded in place onto the shell. In another embodiment, the unique identifier is located on the mandrel surface itself in the form of relief such that the identifier becomes molded into the shell. In another embodiment, the unique identifier is placed on the shell between dippings which form multiple layers of the shell such that the unique identifier is located between such layers.

In some embodiments of the invention, the unique identifier comprises an insert that is first placed in the mold before each and every part of the assembly is molded. In this way, the unique identifier becomes an integral part of the molded assembly.

In one embodiment of the invention, each component of the assembly receives a unique identifier upon its own formation. The presence of such identifying labels on each component from their inception provides maximum traceability of the component back to the point of manufacture. Alternatively, identifying labels may be placed on less than all of the components of the assembly, for example, on just the primary components of the assembly. For example, filled prosthetic implants of the invention may include an identifying label on the shell as soon as it is formed, or during process of formation, and may also be placed on the sprue hole patch and/or on a fill valve if the implant includes such a component.

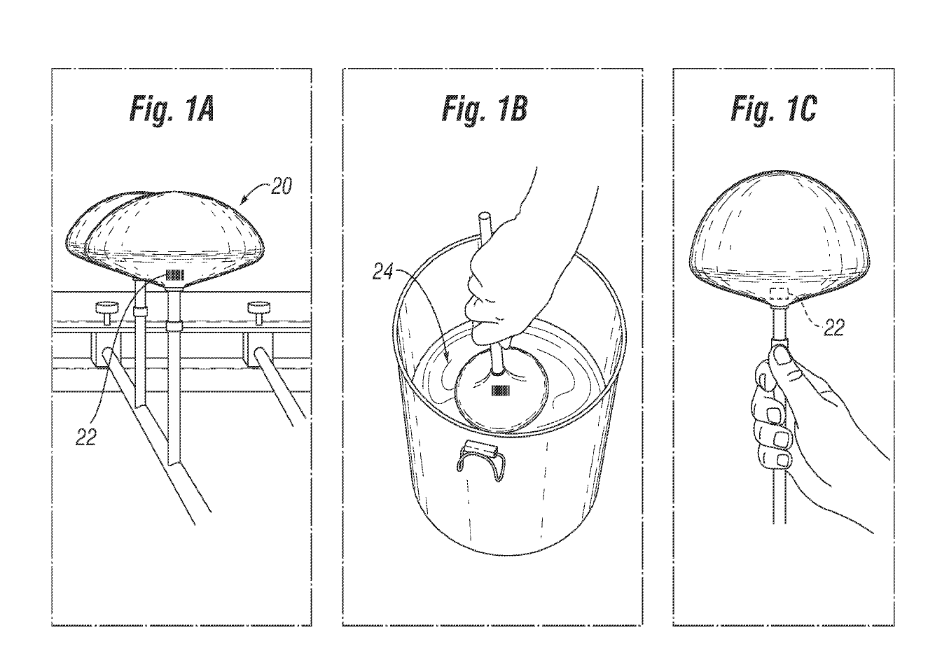

FIGS. 1A-1C illustrate a process of dip-molding the shell of a breast implant prosthesis in accordance with an embodiment of the invention. As seen in FIG. 1A, a mandrel or dip mold 20 having the shape of the interior of a finished shell features a unique identifier label 22, in this case, a bar code. The mold 20 is mechanically or manually dipped into a dispersion 24, for example, a silicone dispersion, as seen in FIG. 1B, so that a layer coats the entire mold. One or more of these dippings are followed by reversion to an upright position as seen in FIG. 1C so that the liquid dispersion evenly coats the mold. As will be explained below, the identifier label 22 may be embedded within the finished shell between layers of the dispersion. Therefore, the label 22 may be introduced to the manufacturing process between successive dippings. The label may include information that can be read by a scanner through the shell.

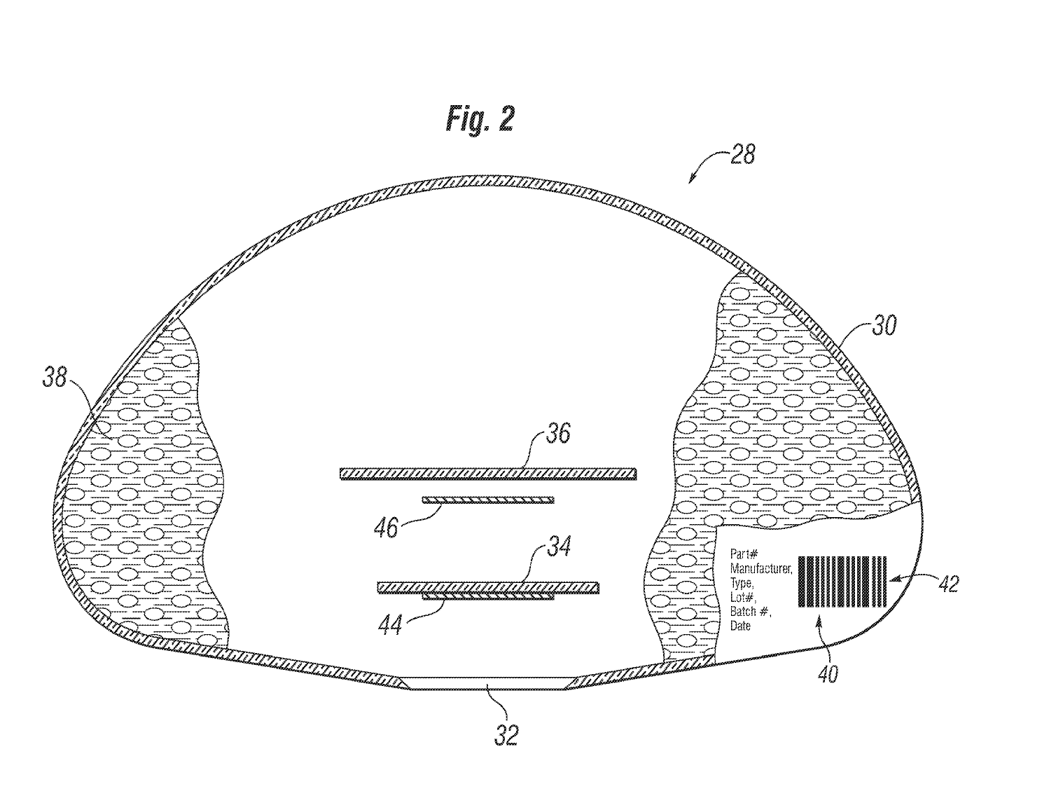

FIG. 2 shows an exemplary filled breast implant prosthesis 28 formed by a process such as the above-described mandrel dipping process. An outer silicone elastomer shell 30 has an anatomical configuration, in this case matching the breast, and comes off a mold with a shell hole 32. In the illustrated embodiment, a patch over the shell hole 32 includes an uncured portion 34 directly over the hole and a cured portion 36 covering that and adhered to the inner surface of the shell 30. Ultimately, the patch is cured, and then the hollow interior of the shell 30 is filled with an appropriate gel 38 via a needle hole in the patch. The silicone gel is supplied as a two-part liquid system with a primary gel component and a cross-linking component. The needle hole in the patch is then sealed with a silicone adhesive and the implant oven cured to achieve cross-linking of the gel.

A unique identifier in the form of a label may be provided within or on the shell 30. For instance, the identifier may be laser etched on a surface of the shell 30. The specific shell or assembly identification information, such as Part #, Lot #, Size, Style, Manufacturer, etc., as indicated at 40, could all be directly laser etched on the outside of the shell. Alternatively, or in addition, the same information may be incorporated into a bar code 42 that makes automatic identification though a scanner possible.

In one embodiment, the shell 30 includes a non-ferromagnetic or weakly ferromagnetic metal such as TiO₂ blended therein that enhances the visibility of such a label, as the metal at the surface heats up and fuses to create visible lines. Moreover, the metallic quality of the label enables imaging thereof in vivo. See Yacoub et al., U.S. Provisional Patent Application Nos. 61/106,449 and 61/106,458, both filed on Oct. 17, 2008, and commonly assigned herewith. The entire disclosure of each of these patent applications is incorporated herein by this specific reference.

In another embodiment, the unique identifier comprises a semiconductor chip that can be read using a scanner or other radio frequency device.

Still with reference to FIG. 2, a second unique identifier label 44 may be provided on the exterior portion 34 of the patch, or an identifier label 46 may be embedded between the layers 34, 36 of the patch. Providing the shell identifier label as well as a patch identifier label facilitates tracing of the manufacturing history of the implant as a whole as well as these separate components of the implant.

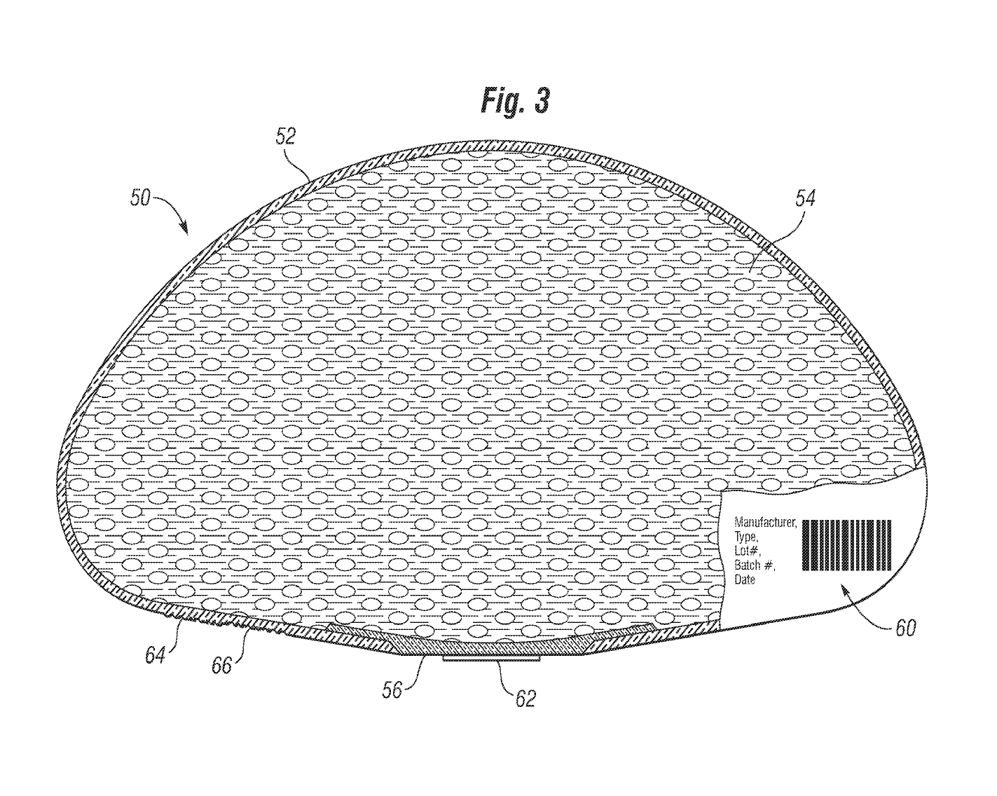

FIG. 3 illustrates an alternative filled breast implant 50 comprising a textured envelope or shell 52 formed for example, by a conventional dip-molding process on a mandrel, such as described above. A fluid 54 such as a silicone gel fills an internal cavity defined within the shell 50. A patch 56 flush with the shell exterior covers a manufacturing hole, though other configurations such as a fluid-adjustment valve or other patch may be substituted. The breast implant 50 features a shell identifier label 60 and a patch identifier label 62, as described above. As explained, the shell identifier label 60 may feature an exterior bar code or characters indicating various characteristics of the component or assembly. For example, the shell identifier label 60 may include specific information on the characteristics of the fluid 54 within the shell 52.

The shell identifier label 60 may be in two- or three-dimensional form. For example, an etched or printed bar code on the exterior of the shell 52 exemplifies a two-dimensional form. (Technically, an etched or laser-etched region is three-dimension on the miniscule level, but for all intents and purposes it appears as a printed 2-D region). FIG. 3 also illustrates an alternative three-dimensional unique identifier 64 comprising an embossed region on the exterior of the shell 52. The embossed region may include information such as shown in the identifier label 60, or may be in another format. Alternatively, a unique identifier 66 may be formed by a series of three-dimensional imprints or indentations. Either identifier 64, 66 may be formed by a reverse image on the shell mold, or may be laser etched.

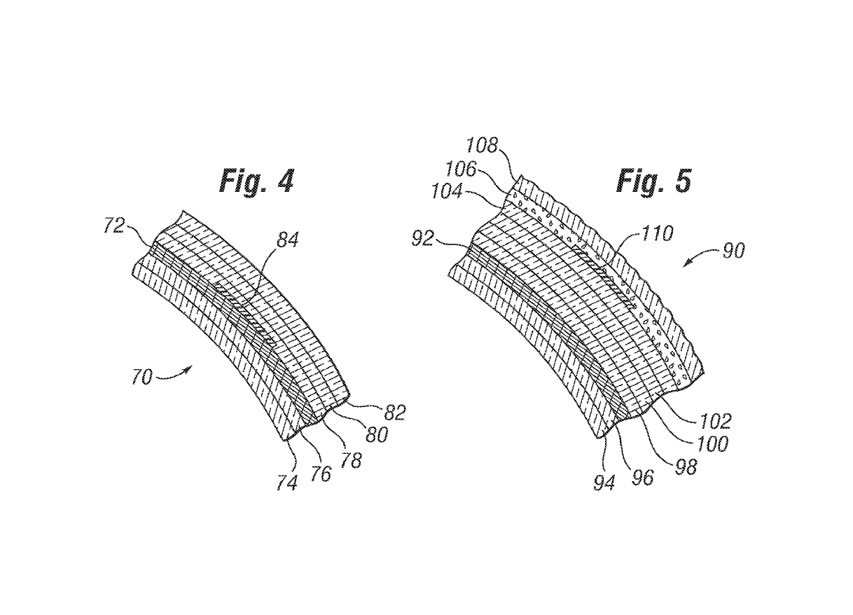

FIG. 4 illustrates in cross-section a layered portion of a smooth-walled implant shell 70. The shell 70 includes an inner primary barrier layer 72, two base coat layers 74, 76 radially inward from the barrier layer, and three further base coat layers 78, 80, 82 outside of the barrier layer. It should be understood that a single inner or outer layer may be used. The thickness of the implant wall may vary but an exemplary average thickness is about 0.456 mm (0.018 inches). The thickness of the barrier layer 72 is typically about 10% of the total wall thickness, or between about 0.025-0.051 mm (0.001-0.002 inches). A unique identifier label 84 is provided between two of the aforementioned layers, in this case between the barrier layer 72 and the first outer base coat layer 78. As mentioned, the identifier label 84 may be positioned on the mandrel subsequent to dipping one of the layers, in this case the barrier layer dispersion 72.

FIG. 5 illustrates in cross-section a layered portion of a textured implant shell 90, which features an inner barrier layer 92, two inner base coat layers 94, 96, and three outer base coat layers 98, 100, 102. Outside of the outer base coat layers, a tack coat layer 104, a layer of textured crystals 106, and an overcoat layer 108 are provided. The absolute thickness of the barrier layer 40 is desirably the same as the smooth-walled version, though the overall thickness of the textured implant wall is somewhat greater because of the extra layers. A unique identifier label 110 is provided between two of the aforementioned layers, in this case outside of the outer base coat layers 98, 100, 102, and a tack coat layer 104, and within the textured outer surface defined by the textured crystals 106 and overcoat layer 108. As mentioned, the identifier label 110 may be positioned on the mandrel subsequent to dipping one of the layers, in this case the tack coat layer dispersion 104.

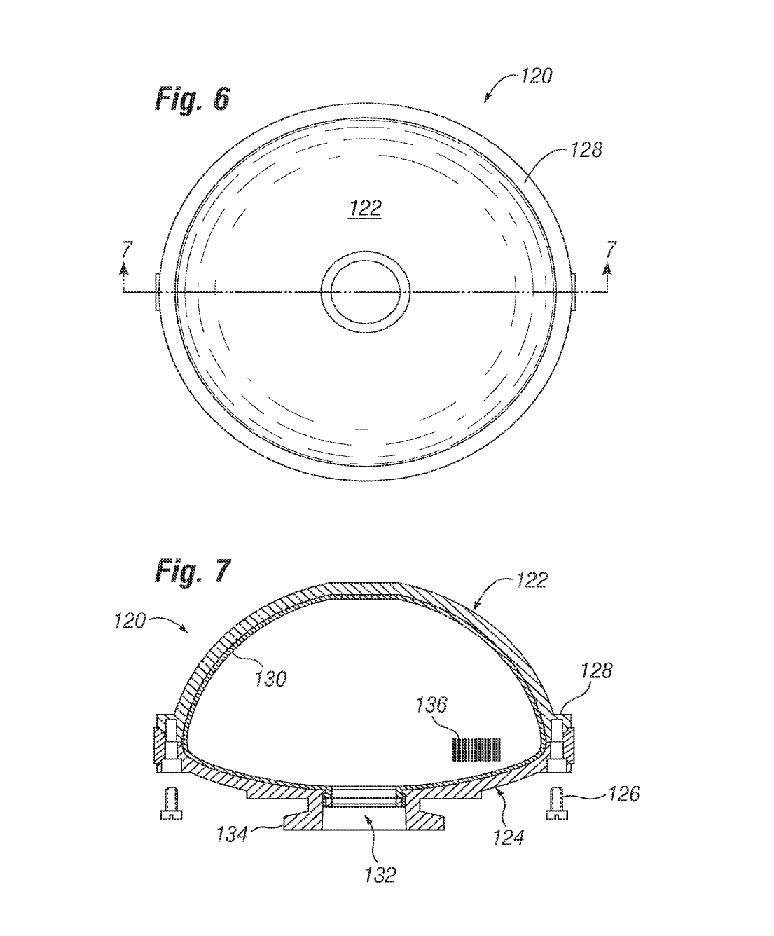

FIG. 6 illustrates an exemplary mold 120 for a rotational molding system that may be used to apply a unique identifier to a prosthetic implant shell formed therein. The mold 120 comprises a top mold piece 122 and bottom mold piece 124 held together by bolts 126 across respective flanges 128, and an inner liner 130 illustrated in cross-section in FIG. 7. The presence of the inner liner 130 enables implant shells to be formed without a seam that otherwise would result at the intersection of the two mold pieces 122, 124. Desirably, the mold pieces 122, 124 are formed of a metal such as aluminum, and the inner liner 130 is formed of a non-adherent or lubricious material such as Teflon, for instance ETFE (ethylene-tetrafluoroethylene).

The mold 120 includes a relatively large circular opening 132 within a lower flange 134 into which projects a sprue tube (not shown) for passage of materials to enter into the mold 120, and for solvents or other gases to escape. Although the mold 120 may be used to form a multi-layered shell, in some embodiments, the mold is used to form a single layer shell. It should be understood, however, that a single layer shell may be formed in multiple steps by a sequence of thin layers such that the finished product exhibits no distinct layers and the entire shell wall is homogenous or uniform in composition.

A unique identifier 136 is shown positioned on an inner surface of the inner liner 130. Again, the unique identifier may be a label or plate that adheres to the exterior of the shell as it is being formed, or may be reverse image raised or indented regions that form in the shell an identifier such as the embossed area 64 or indented region 66 of FIG. 3. In some embodiments, the identifier is located on a separate label or plate that is introduced to the interior of the inner liner 130 before each shell is molded.

In one embodiment, a patch including the identifier is molded in place while the shell is being molded. The patch may be used to cover or patch a hole in the unfinished shell.

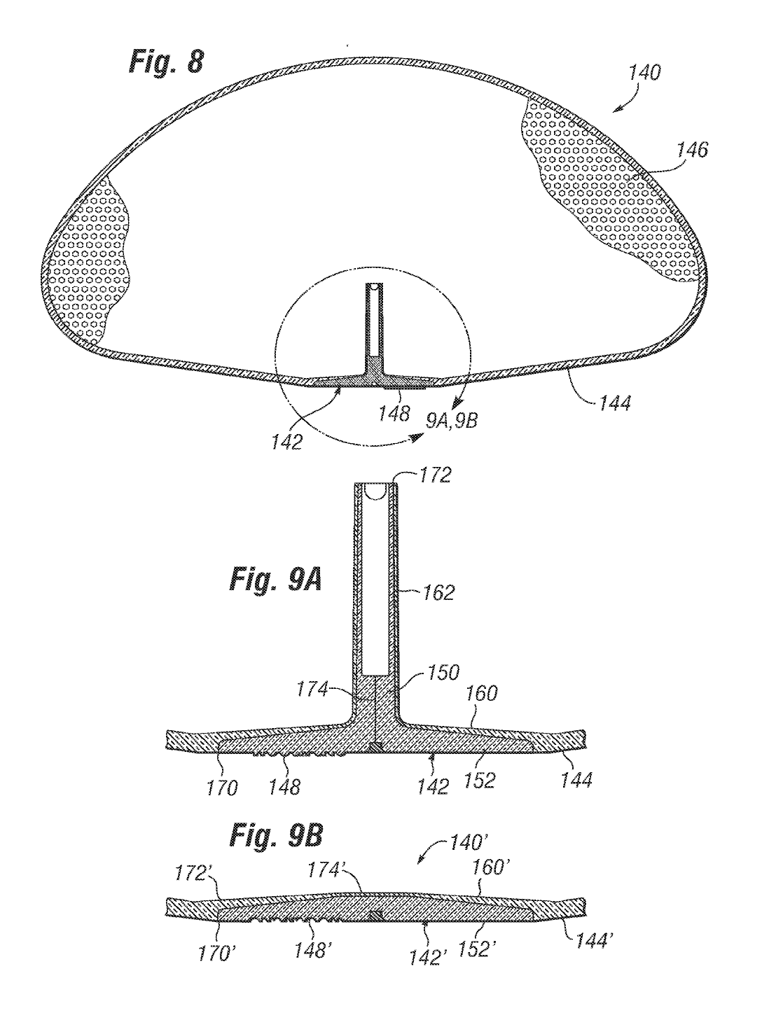

For instance, FIG. 8 is a cross-sectional view through an exemplary gel-filled breast implant prosthesis 140 comprising a shell 144 and a molded-in-place patch 142. The prosthesis 140 is filled with a suitable filler 146, for example, a gel, for example, a silicone gel.

The patch 142 provides a reinforced access region on the surface of the prosthesis 140 for passage of one or more implements from the exterior to the interior. For instance, a rotational mold process as described in co-pending U.S. Provisional Application Ser. No. 61/038,919, filed Apr. 28, 2008, entitled FLUSH PATCH FOR ELASTOMERIC IMPLANT SHELL and expressly incorporated by reference herein, desirably utilizes such a patch 142 as a reinforced conduit through which inserts two silicone dispersion tubes as well as a vent tube, and typically another tube for filling the implant with silicone gel.

Prior to molding the patch 142 in place with the shell 144, a unique identifier 148 is applied. The unique identifier 148 may be any of the configurations described herein, such as a separate label as shown, or an embossed or printed region. In some embodiments, the identifier comprises a laser-etched identifier. The information provided on the patch identifier 148 may include, as before, a serial number, a bar code, a transponder, or other means for human or machine-readable output. Once integrated with the shell 144 as a single unit (a hollow implant), the patch 142 provides information about the source materials and prior process steps, as well as provides subsequent traceability to the implant. For instance, the precise parameters of further finishing and gel-filling steps can be documented and linked to the unique identifier 148. In one scenario, the particular source, temperature, physical makeup (e.g., single layer, wall thickness), chemical makeup, etc. of silicone used to form one implant shell may differ from the next one, which can be traced by referencing process information recorded for the unique identifier 148.

FIG. 9A is a detailed view of the interface between the patch 142 and the shell 144. The patch 142 includes a stem 150 projecting directly radially into the interior of the shell 144 and an outward flange 152 generally conforming to and forming a continuation of the exterior shape of the shell 144. The material of the shell 144 extends over the internal surface of the flange 152 at ring 160, extending at least to the stem 150. In some embodiments, the material of the shell 144 extends in a tube 162 around the stem 150. Because the patch flange 152 increases in radial thickness from its periphery toward its center, the wall thickness of the ring 160 tapers thinner from the main part of the shell 144 to the tube 162. The patch flange 152 may have a uniform thickness along the stem 150.

The unique identifier 148 is shown in FIG. 9A as raised embossed region, which can be formed by a mold that creates the patch 142. In some embodiments, the patch die cut from one or more sheets of silicone material. The unique identifier 148 could be printed, molded, stamped, adhered, or otherwise indicated on the patch 142, in numerous ways. The advantage of labeling the patch 142 which is then molded-in-place with the shell 144 is the relative simplicity of manipulating the patch as opposed to the shell.

By introducing the patch 142 during the process of molding the shell 144, rather than applying the patch to the shell aperture afterwards, the patch integrates with the casting material flowing over and around, thus producing a flush surface both inside and out. In particular, an external surface of the prosthesis including a circular interface line 170 at a flush butt joint between the patch 142 and shell 144 has no ridges or other surface irregularities. Likewise, an internal surface of the prosthesis in the area of the patch 142 has no surface irregularities, and in particular the boundary between the patch 142 and shell 144 is relocated to the radially inner end 172 of the stem 150.

In the shown embodiment, the shell 144 has a substantially contiguous and consistent wall except in an access region across which the patch 142 extends. That is, the access region interrupts the generally constant thickness shell wall. The patch 142 provides an access medium or port through which tubes or other instruments may be inserted into the inner cavity of the shell 144. In the access region, the material of the shell thins to form the ring 160 over the internal surface of the flange 152 and the tube 162 around the stem 150. Because the material of the shell 144 does not cover the open top of the stem 150, an aperture through the shell technically exists, though not the same type of aperture as previously seen with prior art shells. Indeed, in an alternative version in FIG. 9B, the shell may not even have an aperture, and the patch in that case does not cover anything but rather parallels, supports, or is juxtaposed against the thinned access region to provide the access port. In this sense, therefore, the term “patch” is sort of a misnomer, but will be retained for the sake of familiarity.

The stem 150 of the patch 142 may be utilized to help prevent clogging of tubes inserted into the cavity of the mold. For example, a vent tube may extend through a channel 174 (FIG. 9A) in the patch 142 and continue into the mold cavity through the inner end 172 of the stem 150. The silicone dispersion that may at times aggregate near the patch 142 is prevented from entering and potentially clogging the vent tube by virtue of imposition of the upstanding stem 150. The channel 174 also provides an avenue through which a gel-filling tube (not shown) may be introduced after the shell 144 and patch 142 are molded together. For instance, a gel, such as silicone gel 146 shown in FIG. 8, may be injected through a tube inserted through the channel 174. However, instead of providing a pre-formed channel 174, the patch 142 may be made of a self-sealing material or be otherwise configured to be self-sealing.

FIG. 9B illustrates a portion of a soft implant prosthesis 140′ that incorporates a low-profile flush patch 142′ having a unique identifier 148′. The patch 142′ includes an outwardly extending flange 152′ but differs from the above-described patch 142 by eliminating the radially extending stem, and instead has a substantially flat disk shape. The patch 142′ molds in place so that the surrounding shell 144′ again meets flush in a butt joint with the outward flange 152′ to form a smooth exterior surface interface 170′. The material of the shell 144′ also flows over the inner face of the patch 142′ to form a cap 160′ that completely eliminates any internal boundary between the patch and shell. The region 172′ of the shell 144′ adjacent and inward with respect to the outer edge of the patch flange 152′ is smooth, and the thickness of the shell 144′ at that point entirely covers and cushions any potential tactile discontinuity presented by the edge of the flange. There are certainly no sudden surface steps inside and outside the patch periphery, as in the prior art. A self-closing channel 174′ through the patch 142′ again provides a passage for insertion of a vent or gel fill tube, and a small plug 176′ fills a small well at the outlet of the channel after formation of the implant 140′.

The unique identifier 148′ shown in FIG. 9B comprises an indented or laser-etched region. The advantage of laser-etching the patch 142 prior to molding-in-place with the shell 144 is the flexibility of laser etching in conjunction with ease of manipulating the patch. For instance, a manufacturing facility may store a supply of non-labeled patches and laser-etch them in the assembly flow just prior to merging with the shell. Alternatively, the unique identifier 148′ may be provided on the patches well before the step of molding with the shell, and simply recorded at the time of assembly for future reference. Simply providing a unique serial number (or bar code, for instance) for each molded implant separately permits all of the material and process parameters for that particular implant to be recorded and referenced for full traceability during the entire process and afterward. A finished prosthesis that has been packaged for sale, and perhaps implanted, can, years later, be examined and specific data concerning its origin retrieved from the manufacturer's records. This is a great advantage until now unavailable in this field.

For breast implants, the formed implant of the shell 144 and patch 142 is ready for further assembly or processing consistent with the usual manner in creating a final breast implant product. For example, the implant is filled with a filler material of silicone gel or other biocompatible gel material well known to those of skill in the art, such as gel 146 shown in FIG. 8.

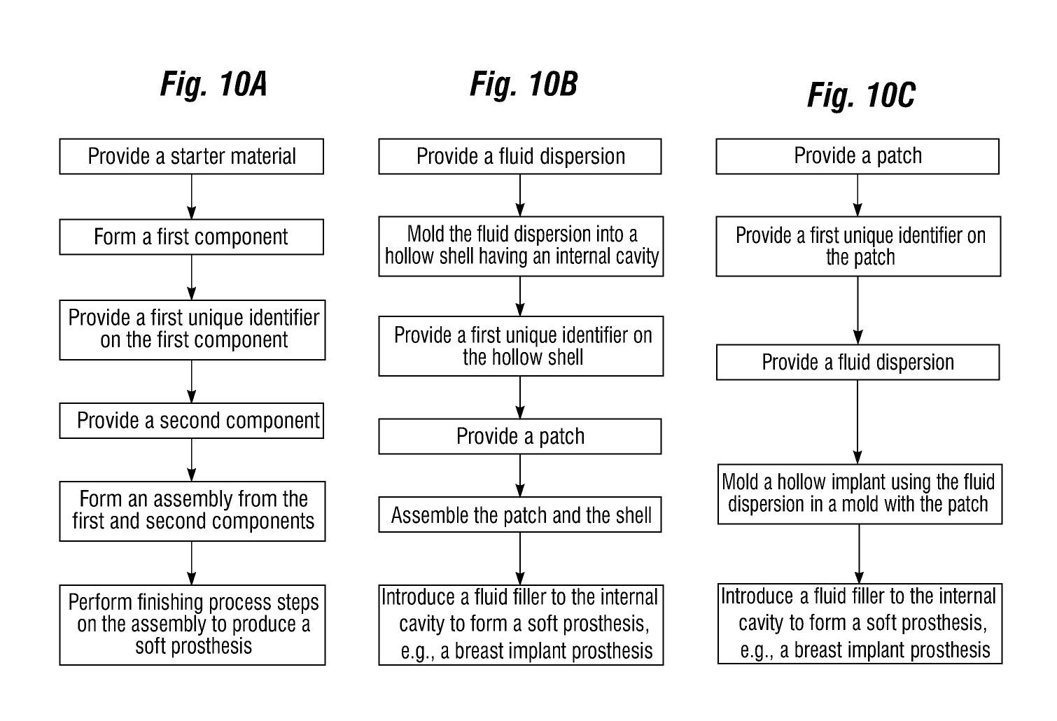

FIG. 10A is a flow chart that shows an exemplary process for applying a unique identifier label to a soft implant prosthesis, while FIGS. 10B and 10C are similar flow charts for soft fluid-filled implants. While the present invention is especially useful in the manufacture of soft fluid-filled implants, such as breast implants, those of skill in the art will recognize that the same principles may apply to other soft prosthetic implants. The flowchart of FIG. 10A thus provides general procedural steps that are embodied for a breast implant, for example, in FIGS. 10B and 10C.

Although the invention has been described and illustrated with a certain degree of particularity, it is understood that the present disclosure has been made only by way of example, and that numerous changes in the combination and arrangement of parts can be resorted to by those skilled in the art without departing from the scope of the invention, as hereinafter claimed.

What is claimed is:

1. A process for forming a soft implant prosthesis, comprising:
providing a fluid dispersion;
molding the fluid dispersion to form a hollow shell having an internal cavity; and
providing an identifier on the implant for tracing the manufacturing history of that implant distinct from other implants, the identifier being applied to the shell during the step of molding the fluid dispersion.

2. The process of claim 1, wherein the step of providing an identifier on the implant comprises providing a unique identifier.

3. The process of claim 1, wherein the step of providing an identifier comprises forming a three-dimensional embossed or indented area on the implant.

4. The process of claim 1, wherein the step of providing an identifier on the implant comprises forming a two-dimensional label on the implant.

5. The process of claim 1, wherein the identifier is embedded between layers of the shell.

6. The process of claim 1, wherein the label is embedded under outer texturing layers of an implant shell wall.

7. The process of claim 1, wherein the identifier is molded-in-place to the shell while the shell is formed from the fluid dispersion.

8. The process of claim 1 wherein the patch with the identifier is molded-in-place into the shell.

9. A soft implant prosthesis made by the process of claim 1.

10. A process for making a breast implant, the process comprising:
providing a silicone fluid dispersion;
molding a breast implant shell by applying the fluid dispersion to a breast implant mandrel;
providing a silicone patch having an identifier for tracing the manufacturing history of the breast implant; and
molding the patch in place to the shell during the step of molding the shell.
